(19) United States
(12) Patent Application Publication
(10) Pub. No.: US 2022/0054660 A1
(43) Pub. Date: Feb. 24, 2022

(54) NANOPARTICLES AND COMPOSITIONS FOR BIOLOGICAL IMAGING BASED ON X-RAY ATTENUATION

(71) Applicant: KOOKMIN UNIVERSITY INDUSTRY ACADEMY COOPERATION FOUNDATION, Seoul (KR)

(72) Inventors: Sanggyu YIM, Seoul (KR); Ilhwan RYU, Seoul (KR); Jee-Yeon RYU, Seoul (KR)

(73) Assignee: KOOKMIN UNIVERSITY INDUSTRY ACADEMY COOPERATION FOUNDATION, Seoul (KR)

(21) Appl. No.: 17/010,861

(22) Filed: Sep. 3, 2020

(30) Foreign Application Priority Data
Aug. 19, 2020 (KR) 10-2020-0103976

(51) Int. Cl. A61K 49/04; A61K 49/00

(52) U.S. Cl. CPC A61K 49/0423; B82Y 5/00; A61K 49/0093

(57) ABSTRACT

The present invention relates to an X-ray attenuation-based biometric imaging technology and includes a core structure comprising an X-ray attenuating material; and a shell layer formed on the core structure and made of a material having biocompatibility and non-reactivity in vivo, and it has the effect of enabling simple and rapid cancer diagnosis by plain X-ray imaging, such as those used for non-invasive chest X-ray.

### CROSS-REFERENCE TO RELATED APPLICATION

This application claims priority to and the benefit of Korean Patent Application No. 10-2020-0103976 filed in the Korean Intellectual Property Office on Aug. 19, 2020, the entire contents of which are incorporated herein by reference.

### BACKGROUND OF THE DISCLOSURE

### 1. Field of the Disclosure

The present invention relates to a nanoparticle technology for biological imaging based on X-ray attenuation.

### 2. Description of the Related Art

The incidence and mortality of cancer are steadily growing, and cancer is one of the major barriers to increasing life expectancy worldwide. Early diagnosis of cancer leads to increased chances for curative treatment and improved survival against most cancers, especially those that are aggressive or have no early symptoms. Although non-invasive anatomical imaging technologies such as magnetic resonance imaging (MRI), computed tomography (CT) and ultrasound (US) imaging have become indispensable to cancer diagnosis, they have their respective drawbacks such as long acquisition time, high radiation exposure and low resolution at increased depth. Furthermore, the contrast issues of these tomographical techniques significantly limit their use if the morphology of tumor is similar to that of healthy tissues; thus they are less able to detect early or subtle changes that occur in cancers such as pancreatic cancer and colon cancer.

Alternatively, the technology of using non-invasive optical imaging with fluorescent molecules or nanoparticles (NPs) designed to specifically bind to cancer has steadily advanced. Researches on the fluorescence imaging using near-infrared (NIR), especially second-NIR (NIR-II, 900-1700 nm), light have rapidly increased since it provides longer tissue penetration and better temporal and spatial resolution compared to visible or ultra-violet (UV) light due to its reduced tissue absorption, scattering and autofluorescence. Nevertheless, intrinsic problems of the fluorescence-based approaches, such as limited penetration depth of the absorption and emission light and a trade-off between the depth and resolution, are unavoidable. The issues on bio-toxicity, low quantum yield and insufficient detection systems also restrict their clinical adoption.

In contrast, X-ray-based imaging is fast, easy to use and has almost no limitation in penetration depth. Specifically, compared to other X-ray techniques such as CT, plain X-ray imaging such as those used in chest X-rays has additional advantages including lower cost, much less exposure to radiation and easier accessibility.

Therefore, there is a need to develop nanoparticles capable of real-time in vivo imaging including early diagnosis of tumors by non-invasive plain X-ray examination and research into in vivo imaging technology using the same.

### PRIOR ARTS DOCUMENTS

### Non-Patent Documents

1. Nat. Biomed. Eng. 1, 697-713 (2017) (published on Sep 12, 2017)

### SUMMARY OF THE DISCLOSURE

An object of the present invention is to provide nanoparticles for biological imaging based on X-ray attenuation, which are capable of non-invasive in vivo imaging through plain X-ray imaging and a method of preparing the same.

Also, another object of the present invention is to provide a composition for biological imaging based on X-ray attenuation comprising the nanoparticles.

In addition, another object of the present invention is to provide a method of an X-ray attenuation-based biological imaging comprising the nanoparticles.

In order to achieve the above object, the present invention provides nanoparticles for biological imaging based on X-ray attenuation comprising: a core structure comprising an X-ray attenuating material; and a shell layer formed on the core structure and made of a material having biocompatibility and non-reactivity in vivo.

Also, the present invention provides a composition for biological imaging based on X-ray attenuation comprising the nanoparticles for biological imaging based on X-ray attenuation as an active ingredient.

In addition, the present invention provides a method of biological imaging based on X-ray attenuation comprising the steps of reacting the nanoparticles for biological imaging based on X-ray attenuation with a biological sample; and observing an image by X-ray imaging.

Furthermore, the present invention provides a method of preparing nanoparticles for biological imaging based on X-ray attenuation comprising the steps of preparing an X-ray attenuating material precursor solution (Step 1); preparing a core structure by adding the solution of the step 1 to the core precursor solution (Step 2); annealing the core structure of the step 2 (Step 3); and preparing nanoparticles having a core precursor-shell layer by adding annealed core structure of the step 3 to a shell precursor solution (Step 4).

### BRIEF DESCRIPTION OF THE DRAWINGS

FIG. 1 shows plain X-ray imaging of cancer using cesium lead bromide (CPB) quantum-dot (QD) scintillators. (A) Schematic illustration of cancer detection by plain X-ray inspection. (B) Structure of CPB—SiO₂©SiO₂—Ab nanoparticles (NPs). (C and D) Transmission electron microscopy (TEM) images of CPB—SiO₂©SiO₂ NPs at different magnifications. (The CPB QDs encased in the SiO₂ NP are marked with green arrows in (C)). (E) Photographs of CPB—SiO₂©SiO₂ NPs powder under UV irradiation and daylight (inset).

FIG. 2 shows spectroscopic investigation of CPB—SiO₂©SiO₂ NPs. (A to C) FT-IR spectra (A) and X-ray photoelectron spectra for O 1 s electrons (B and C) of the CPB—SiO₂ NPs before and after annealing at 150° C. for 2 h. (D) Comparison of Si-OH related spectroscopic intensities before and after annealing. (E and F) Variations of Pb concentration estimated by inductively coupled plasma (ICP) measurements (E) and photoluminescence (PL) intensity (F) over time for aqueous CPB-SiO₂@SiO₂ NP solution (1 mg/ml).

FIG. 3 shows spectroscopic investigation of CPB-SiO₂ NPs. Relative photoluminescence (PL) intensities over time for aqueous solution (1 mg/ml) containing CPB-SiO₂ NPs unannealed and annealed at 150° C. for 2 h.

FIG. 4 shows morphology and fluorescence characteristics of CPB-SiO₂@SiO₂ NPs. (A, B) Scanning electron microscopy (SEM) images of CPB-SiO₂@Si0₂ NPs taken at different magnifications. (C) Photoluminescence (PL) spectra of CPB-SiO₂ NP and CPB-SiO₂@SiO₂ NP solutions.

FIG. 5 shows depth profile X-ray photoelectron spectroscopy (XPS) measurements. Variation of atomic contents of O 1 s, Si 2 p and Pb 4 f core electrons for CPB-SiO₂©SiO₂ NP-deposited films, plotted as a function of etching time (film depth).

FIG. 6 shows X-ray attenuation characteristics of CPB-SiO₂@SiO₂ NPs. (A) X-ray images of CPB-SiO₂@SiO₂ NPs taken at various tube potentials. (The thicknesses of NP-containing cylinder are 0.5, 1.0 and 2.0 cm from left to right.) (B) X-ray images of the NPs placed under muscle and bone taken at the tube potential of 50 kVp. (The thicknesses are 0.5, 1.0 and 2.0 cm from left to right.) (C) X-ray images of the smaller amount of NPs placed under muscle and bone. (The tube potential is 50 kVp, and the amounts of NPs are 1, 3, 5, 10 and 20 mg from left to right.) The contrast resolution and SBR values are also presented.

FIG. 7 shows In vitro cellular uptake of CPB-SiO₂@SiO₂ NPs and in vivo X-ray cancer imaging. (A) Confocal laser-scanning microscopy (CLSM) images of Panc-1 cells treated with 0.5 mg/mL CPB-SiO₂@SiO₂-Ab NPs (denoted as CPB-S@SiO₂) for 24 h. (The nuclei of the cells were stained with DAPI (blue) and the NPs are identified with green.) (B) Cellular uptake efficiencies determined by photoluminescence (PL) of Panc-1 cells treated with various concentrations of CPB-S@SiO₂-Ab NPs for 24 h. (C) PL spectra of 1.0 mg/ml CPB-S@SiO₂-Ab NP solution and Panc-1 cells treated with this solution. (D) Cell viabilities determined by WST-1 assay. (Cells were treated with various concentrations of CPB-S@SiO₂-Ab NPs. Error bars represent mean ±S.D. (n=3).) (E) In vivo photographic, X-ray and X-ray plus fluorescence overlay images of pancreatic tumor-bearing mice after 2 h intravenous injection of CPB-S@SiO₂-Ab NPs (10 mg/kg). (Yellow circles indicate the area where the pancreatic tumor was grown.) (F) Real-time in vivo X-ray and X-ray plus fluorescence overlay images at various time points after the injection of the NPs (10 mg/kg). (White arrows indicate the tumor area.)

FIG. 8 shows biodistribution of injected CPB-SiO₂@SiO₂-Ab NPs. (A, C) Ex vivo fluorescence imaging of various organs dissected 2 h (A) and 10 days (C) after CPB-SiO₂@SiO₂-Ab NP injection. (B) Relative fluorescence intensities of organs dissected 2 h after NP injection compared to those of organs dissected from mice without NPs injection. (Error bars represent mean ±S.D. (n=3 mice per group).)

FIG. 9 shows toxicity evaluation of CPB-SiO₂@SiO₂-Ab NPs. Microscopic images of hematoxylin and eosin (H&E) staining of organs (liver, spleen, stomach, intestine, kidney and testis) from mice untreated and treated with CPB-SiO₂@SiO₂-Ab NPs. (Scale bar represents 200 μm.)

FIG. 10 shows evaluation of toxicity of CPB-SiO₂@SiO₂-Ab NPs according to body weight change. Body weights measured over 14 days for control mice and mice injected with CPB-SiO₂@SiO₂-Ab NPs (10 mg/kg). (Error bars represent mean ±S.D. (n=3 mice per group).)

FIG. 11 shows the synthesis results of various types of CsPbX₃-SiO₂@SiO₂ NP powders according to the halogen composition, (A) a photo of each powder under UV irradiation and (B) a photoluminescence measurement result of each powder.

### DETAILED DESCRIPTION OF THE EMBODIMENTS

Hereinafter, the present invention will be described in detail.

The present inventors have prepared nanoparticles comprising an X-ray attenuating material such as lead bromide (CsPbBr₃, CPB) perovskite quantum dots (QD) having excellent biosafety and stability and completed the present invention by confirming that cancer can be imaged by the X-ray attenuation reaction of quantum dots only with plain X-ray imaging to be useful for early diagnosis of cancer, as well as in vivo imaging.

The present invention provides nanoparticles for biological imaging based on X-ray attenuation comprising: a core structure comprising an X-ray attenuating material; and a shell layer formed on the core structure and made of a material having biocompatibility and non-reactivity in vivo.

At this time, the X-ray attenuating material may comprise an ABX₃ perovskite structure material, which is a quantum dot material having an average diameter of 5 to 15 nm, and the shell layer may comprise at least one selected from the group SiO₂, TiO₂, ZnO, ZrO₂ and Al₂O₃.

(The A is selected from the group consisting of Ti, Sr, Ca, Cs, Ba, Y, Gd, La, Fe and Mn, the B is selected from the group consisting of Pb, Sn, Cu, Ni, Bi, Co, Fe, Mn, Cr, Cd, Ge and Yb, and the X is selected from the group consisting of l_{y}Br(_{1-y}), l_{y}Cl(_{1-y}) and Br_{y}Cl(_{1-y}) (0≤y≤1)).

According to an embodiment of the present invention, the nanoparticles according to the present invention can be completely prevented from being decomposed or released by encapsulating an X-ray attenuating quantum dot material with SiO₂.

In addition, the shell layer further may comprise at least one targeting agent selected from the group consisting of enzyme substrates, ligands, amino acids, peptides, proteins, nucleic acid, lipids, cofactors, carbohydrates and antibodies on the surface, but it is not limited thereto, and the targeting agent may increase the absorption rate of the nanoparticles, and specifically target and bind to a biological sample such as cells or tissues in vivo. Furthermore, any antibody may be used as long as the antibody specifically targets only cancer cells and binds to cancer cells.

In addition, the cancer cells may be selected from the group consisting of colon cancer, liver cancer, stomach cancer, breast cancer, colon cancer, bone cancer, pancreatic cancer, head or cervical cancer, uterine cancer, ovarian cancer, rectal cancer, esophageal cancer, small intestine cancer, anal cancer near the anus, fallopian tube carcinoma, endometrial carcinoma, cervical carcinoma, vaginal carcinoma, vulvar carcinoma, Hodgkin's disease, prostate cancer, bladder cancer, kidney cancer, ureteral cancer, renal cell carcinoma, renal pelvic carcinoma, central nervous system tumor, and brain tumor. According to the invention, cancer can be diagnosed by a change caused by X-ray attenuation by plain X-ray imaging, so all cancer cells known to a person skilled in the art can be diagnosed.

In addition, the present invention provides a method of biological imaging based on X-ray attenuation comprising the steps of reacting the nanoparticles for biological imaging based on X-ray attenuation with a biological sample; and observing an image by X-ray imaging.

According to an embodiment of the present invention, the antibody, which is a cancer-specific targeting agent bound to the nanoparticles, attaches the nanoparticles to cancer cells, and when X-rays are irradiated thereto, lead cesium bromide quantum dot scintillator significantly reduces the amount of X-ray photons that transmit cancer cells by emitting bright or fluorescing at the tumor site due to the X-ray attenuation or fluorescence characteristics of the lead cesium bromide quantum dot scintillator so as to diagnose cancer.

In addition, the present invention provides a composition for biological imaging based on X-ray attenuation comprising the nanoparticles for biological imaging based on X-ray attenuation as an active ingredient.

The composition may contain a pharmaceutically acceptable carrier. The pharmaceutically acceptable carrier may include physiological saline, polyethylene glycol, ethanol, vegetable oil and isopropyl myristate, but it is not limited thereto.

In addition, the present invention provides a method of preparing nanoparticles for biological imaging based on X-ray attenuation comprising the steps of preparing an X-ray attenuating material precursor solution (Step 1); preparing a core structure by adding the solution of the step 1 to the core precursor solution (Step 2); annealing the core structure of the step 2 (Step 3); and preparing nanoparticles having a core precursor-shell layer by adding annealed core structure of the step 3 to a shell precursor solution (Step 4).

According to an embodiment of the present invention, a method of preparing the nanoparticle is a new and effective synthesis method for rapid co-synthesis of CPB QD, that is, QD and NP inside SiO₂ NP, and this rapid co-synthesis can lead to CPB QD being trapped inside SiO₂ NPs before its decomposition.

At this time, the X-ray attenuating material precursor is selected from the group consisting of lead bromide (PbBr₂), cesium bromide (CsBr), cesium iodide (Csl), cesium chloride (CsCl), and lead chloride (PbCl₂), and the X-ray attenuating material precursor solution may include a basic catalyst, specifically an ammonia catalyst, but it is not limited thereto.

In addition, the core precursor or the shell precursor may be selected from the group consisting of tetramethyl orthosilicate and tetraethyl orthosilicate, but it is not limited thereto.

In addition, the annealing may be performed for 1 to 3 hours at 100 to 200° C., and stability is improved by reducing the hydroxyl groups on the surface of the nanoparticles by this annealing process.

In addition, it may further include the step of binding a targeting agent to the shell layer of the nanoparticles having the core precursor-shell layer, and the binding the targeting agent to the shell layer of the nanoparticles having the core precursor-shell layer may comprise the steps of modifying the shell layer by reacting the nanoparticles having the core precursor-shell layer with 3-aminopropyl triethoxysilane and 4-(N-maleimidomethyl)cyclohexane-1-carboxylic acid 3-sulfo-N-hydroxysuccinimide ester sodium salt; and conjugating modified nanoparticles with a targeting agent.

The absorption rate of the nanoparticles can be increased by the process of binding such a targeting agent to the shell layer of the nanoparticles, and it can be specifically targeted and bound to a biological sample such as cells or tissues in vivo.

In addition, the present invention provides a novel and effective X-ray attenuation-based in vivo imaging method that can be detected anywhere, such as cells or tissues in vivo, using a lead cesium bromide (CsPbBr₃, CPB) perovskite quantum dot (QD) scintillator. The CPB perovskite QD has excellent ability to convert incident X-ray photons into visible light emission with excellent spatial resolution. However, its use was limited because of its poor stability against moisture and the possibility of releasing toxic Pb compounds.

According to an embodiment of the present invention, the stability problem was solved by introducing silicon dioxide (SiO₂) core/shell nanoparticles (NP) embedded with CPB QD. The core portion was formed by rapid co-synthesis of CPB QD and SiO₂ NP; the shell portion was formed by growing an additional SiO₂ layer outside the synthesized core NP to completely prevent QDs from being released or decomposed. Subsequently, an anti-CD44 antibody (Ab) targeting a cancer cell surface-attached receptor was conjugated to the surface of CPB-SiO₂@SiO₂ NP. A small amount of CPB-SiO₂@SiO₂-Ab NP (2.8 μg based on QD) was injected intravenously into mice with pancreatic tumors of approximately 5 mm in size; Thereafter, a plain X-ray image was taken. The bright white spots from the strong X-ray attenuation of CPB QD at the tumor site gradually became intense and reached the highest intensity in 2 hours after injection. In addition, the stability, X-ray attenuation properties of CPB QD, and bio-distribution and toxicity of QD-containing SiO₂ NPs were evaluated for clinical application.

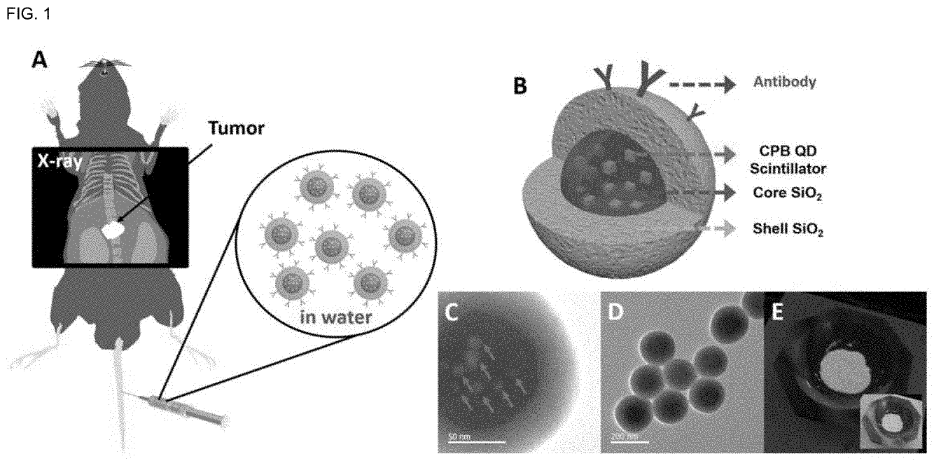

The basic strategy for in vivo imaging of cancer by plain X-ray examination was shown in FIG. 1A and FIG. 1B. SiO₂ NPs containing X-ray scintillation CPB QD were introduced into xenograft mice via intravenous (IV) injection. The cancer-specific antibody conjugated on the NP attaches the NP to the cancer cells, and the internal X-ray scintillation QD significantly reduced the amount of X-ray photons penetrating the cancer cells during X-ray irradiation and thus the dark area appeared bright during imaging. Accordingly, the present invention enables in vivo imaging of cancer by a plain X-ray examination.

Hereinafter, the present invention will be described in more detail through examples. These examples are only intended to illustrate the present invention in more detail, and it will be apparent to those skilled in the art that the scope of the present invention is not limited by these examples according to the gist of the present invention.

### <Reference Example> Materials

Lead bromide (PbBr₂, 99.999%), cesium bromide (CsBr, 99.999%), oleic acid (OA, 90%, technical grade), oleyl amine (OLA, 70%, technical grade), tetramethyl orthosilicate (TMOS, 98%), tetraethyl orthosilicate (TEOS, 98%, reagent grade), 3-aminopropyl triethoxysilane (APTES, 99%) and ammonium hydroxide (NH₄OH, 28%), anti-CD44 (antibody produced in rabbit), 4-(N-maleimidomethyl)cyclohexane-1-carboxylic acid 3-sulfo-N-hydroxysuccinimide ester sodium salt (sulfo-SMCC) and 2-mercapto-ethyl-amine hydrochloride (2-MEA, 98%) were purchased from Sigma-Aldrich and used as received. N,N-dimethylformamide (DMF, 99.5%), toluene (99.7%) and anhydrous ethanol (99.8%) were purchased from DAEJUNG and used without further purification.

### <Example 1>Synthesis of CsPbBr₃-SiO₂@SiO₂ NPs

First, the CPB precursor solution was prepared by adding 0.3 g PbBr₂, 0.17 g CsBr, 1.2 ml OLA and 3.6 ml OA to 20 ml DMF and stirring the mixture at 90° C. until it became transparent. The catalytic amount of 28% aqueous NH₄OH solution was slowly added to the CPB precursor solution. Two millilitres of this ammonia-containing CPB precursor solution was rapidly injected into a 400 μl/100 ml TMOS/toluene solution, followed by stirring for 2 h at room temperature. The synthesized CPB-SiO₂ NPs were collected using centrifugation and were washed with ethanol three times. The obtained powder was annealed at 150° C. for 2 h to remove surface hydroxyl groups. With the addition of NPs to the 1.2 ml/40 ml TEOS/ethanol solution, stirring at room temperature for 1 h, and then injection of 4 ml 28% NH₄OH solution, additional SiO₂ layers were formed on the annealed-CPB-SiO₂ NPs. After 20 h of stirring at room temperature, synthesized CPB-SiO₂@SiO₂ NPs were collected by centrifugation and washed with ethanol three times.

### <Example 2> Synthesis of CsPbl₂Br₁-SiO₂@SiO₂ NPs

The precursor solution was prepared by adding 0.2212 g Pbl₂, 0.08376 g PbBr₂, Csl 0.1248 g, CsBr 0.068 g, 1.2 ml OLA and 3.6 ml OA to 20 ml DMF and stirring the mixture at 90° C. until it became transparent. The catalytic amount of 28% aqueous NH₄OH solution was slowly added to the CsPbl₂Br₁ precursor solution. Two millilitres of this ammonia-containing CsPbl₂Br₁ precursor solution was rapidly injected into a 1600 μl/100 ml TMOS/toluene solution, followed by stirring for 2 h at room temperature. The synthesized CsPbl₂Br₁-SiO₂ NPs were collected using centrifugation and were washed with ethanol three times. The obtained powder was annealed at 150° C. for 2 h to remove surface hydroxyl groups. With the addition of NPs to the 1.2 ml/40 ml TEOS/ethanol solution, stirring at room temperature for 1 h, and then injection of 4 ml 28% NH₄OH solution, additional SiO₂ layers were formed on the annealed-CsPbl₂Br₁-SiO₂ NPs. After 20 h of stirring at room temperature, synthesized CsPbl₂Br₁-SiO₂@SiO₂ NPs were collected by centrifugation and washed with ethanol three times.

### <Example 3> Synthesis of CsPbBr₂Cl₁-SiO₂@SiO₂ NPs

The precursor solution was prepared by adding 0.0134 g CsCl, 0.2936 g PbBr₂, 1.2 ml OLA and 3.6 ml OA to 20 ml DMF and stirring the mixture at 90° C. until it became transparent. The catalytic amount of 28% aqueous NH₄OH solution was slowly added to the CsPbBr₂Cl₁ precursor solution. Two millilitres of this ammonia-containing CsPbBr₂Cl₁ precursor solution was rapidly injected into a 400 μl/100 ml TMOS/toluene solution, followed by stirring for 2 h at room temperature. The synthesized CsPbBr₂Cl₁-SiO₂ NPs were collected using centrifugation and were washed with ethanol three times. The obtained powder was annealed at 150° C. for 2 h to remove surface hydroxyl groups. With the addition of NPs to the 1.2 ml/40 ml TEOS/ethanol solution, stirring at room temperature for 1 h, and then injection of 4 ml 28% NH₄OH solution, additional SiO₂ layers were formed on the annealed-CsPbBr₂Cl₁-SiO₂ NPs. After 20 h of stirring at room temperature, synthesized CsPbBr₂Cl₁-SiO₂@SiO₂ NPs were collected by centrifugation and washed with ethanol three times.

### <Example 4> Synthesis of CsPbBr₁Cl₂-SiO₂@SiO₂ NPs

The precursor solution was prepared by adding 0.0117 g CsBr, 0.2225 g PbCl₂, 1.2 ml OLA and 3.6 ml OA to 20 ml DMF and stirring the mixture at 90° C. until it became transparent. The catalytic amount of 28% aqueous NH₄OH solution was slowly added to the CsPbBr₁Cl₂ precursor solution. Two millilitres of this ammonia-containing CsPbBr₁Cl₂ precursor solution was rapidly injected into a 400 μl/100 ml TMOS/toluene solution, followed by stirring for 2 h at room temperature. The synthesized CsPbBr₁Cl₂-SiO₂ NPs were collected using centrifugation and were washed with ethanol three times. The obtained powder was annealed at 150° C. for 2 h to remove surface hydroxyl groups. With the addition of NPs to the 1.2 ml/40 ml TEOS/ethanol solution, stirring at room temperature for 1 h, and then injection of 4 ml 28% NH₄OH solution, additional SiO₂ layers were formed on the annealed-CsPbBr₁Cl₂-SiO₂ NPs. After 20 h of stirring at room temperature, synthesized CsPbBr₁Cl₂-SiO₂@SiO₂ NPs were collected by centrifugation and washed with ethanol three times.

### <Example 5> Surface Modification and Antibody Conjugation

The synthesized CPB-SiO₂@SiO₂ NPs were dissolved in anhydrous ethanol at a concentration of 5 mg/ml. Excess amount of APTES was added to the solution and then kept overnight at 60° C. CPB-SiO₂@SiO₂ NP surface-amine-functionalized NPs were collected by centrifugation and reacted with sulfo-SMCC for 2 h at room temperature. Separately, anti-CD44 antibodies were incubated with 50 mM 2-MEA for 1.5 h at 37° C. and then, by passing the mixture through a desalting column, separated from excess 2-MEA. Finally, antibody conjugation on the CPB-SiO₂@SiO₂ NPs was performed by mixing at 4° C. for 2 h the maleimide-activated NPs and sulfhydryl groups containing antibodies.

### <Example 6> Uptake Efficiency of CPB-SiO₂@SiO₂-Ab NPs in Vitro

Panc-1 cells were grown in Dulbecco's Modified Eagle's Medium (DMEM, Gibco) supplemented with 10% fetal bovine serum (FBS) and 1% penicillin-streptomycin (Pen-Strep) at 37° C. under 5% CO₂ overnight. To estimate the uptake efficiency of the NPs, the cells were seeded in 60-mm dishes (1×10⁶ cells) and incubated for 24 h at 37° C. in the CPB-SiO₂@SiO₂-Ab NPs-containing phosphate-buffered saline (PBS) solution at various NP concentrations (0.1, 0.2, 0.5 and 1.0 mg/ml). The cells were fixed with formaldehyde and fluorescence emission was measured by PL spectroscopy.

### <Example 7>Cell Viability Assay

For the cell viability test, Panc-1 cells were seeded in 96-well plates and treated with CPB-SiO₂@SiO₂-Ab NP solutions of various concentrations (0.1, 0.2, 0.5 and 1.0 mg/ml in PBS) for various durations (24, 48 and 72 h). Cell viabilities were analyzed using the water-soluble tetrazolium salt (WST-1) assay (Dogen) according to the manufacturer's protocol. WST-1 solution was added to each well and the cells were incubated at 37° C. for 30 min. After the incubation, absorbance at 450 nm was measured using a microplate reader (BioTek).

### <Example 8>Immunofluorescence

Panc-1 cells were seeded in a 4-well chamber slide (1.5×10⁴ cells per well) and treated with CPB-SiO₂@SiO₂-Ab NP solution (0.5 mg/mL) for various durations. The cells were fixed in 4% paraformaldehyde for 15 min, permeabilized with 0.1% Triton X-100 in PBS at room temperature for 15 min and then blocked in 2% BSA at room temperature for 1 h. The cells were stained with DAPI (Abcam) and examined using confocal laser scanning microscopy (CLSM).

### <Example 9> Mouse Xenograft Experiments and Imaging

All animal experiments were performed under the guidelines of the Seoul St. Mary's Hospital animal care and use committee. BALB/c nude mice (Orient Bio) were implanted subcutaneously with Panc-1 (3×10⁶ cells) in matrigel. Tumor growth was monitored until it reached an acceptable size of about 150±30 mm³. 200 μl of CPB-SiO₂@SiO₂-Ab NP solution (1 mg/ml) was injected into the tail vein. Prior to obtaining real-time X-ray images, all mice were anesthetized with isoflurane and medical grade oxygen. To investigate the biodistribution of the CPB-SiO₂@SiO₂-Ab NPs, mice were sacrificed and dissected 2 h or 10 days after NP injection.

### <Example 10> Characterization

The size and shape of the synthesized CPB-SiO₂ and CPB-SiO₂@SiO₂ NPs were characterized by high-resolution transmission electron microscopy (HRTEM, JEM-2100F, JEOL Ltd.) and field emission scanning electron microscopy (FE-SEM, JSM-7610F, JEOL Ltd.). The variation of the hydroxyl groups on the surface of CPB-SiO₂ NPs was characterized by Fourier-transform infrared (FT-IR) spectrometry (Nicolet™ iS™ 50 FTIR Spectrometer, Thermo Fisher) and X-ray photoelectron spectroscopy (XPS, PHI 5000 VersaProbe, ULVAC PHI). The PL spectra of CPB-SiO₂ and CPB-SiO₂@SiO₂ NPs were recorded using a fluorescence spectrophotometer (SM245, Korea Spectral Products and Darsa Pro-5200, PSI Co. Ltd.) with excitation of 405 nm. The release of elemental lead from CPB-SiO₂@SiO₂ NPs was detected by inductivity coupled plasma mass spectroscopy (ICP-MS, NexION 350D, Perkin-Elmer SCIEX). In vivo fluorescence and X-ray images were acquired on an Optical in vivo Imaging System-IVIS Lumina XRMS (PerkinElmer Inc.). Tumor and major organs were dissected and immediately fixed in 10% formalin for 24 h, and then their images were taken with the same IVIS Lumina XRMS. For microscopic assessment, the tissues were embedded in paraffin and sectioned (thickness: 5-15 μm), and the sections were stained with hematoxylin and eosin (H&E).

### <Experimental Example 1> Synthesis of CsPbX₃-SiO₂@SiO₂ NPs

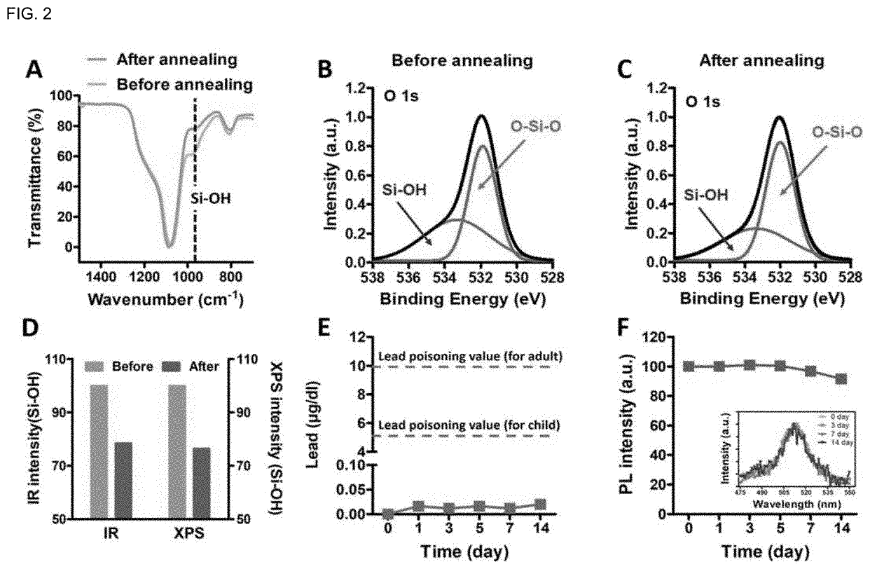

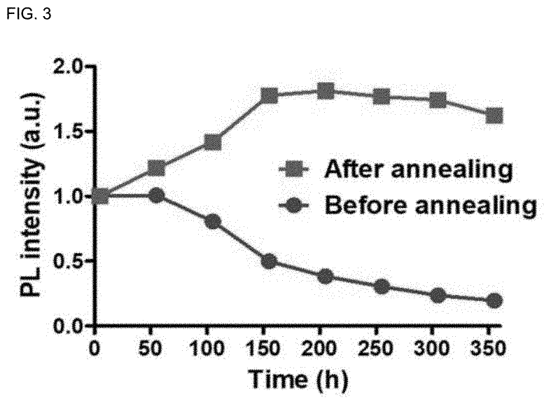

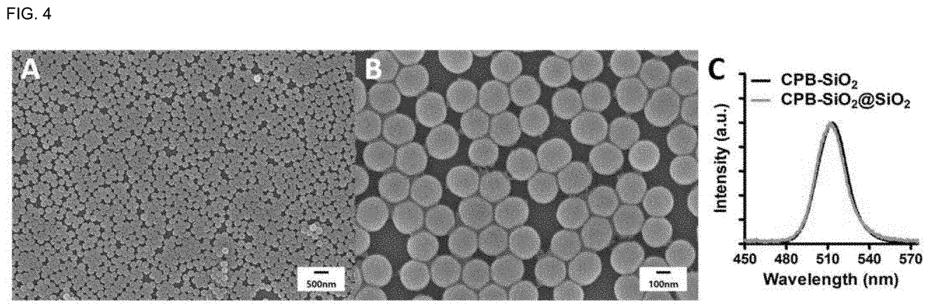

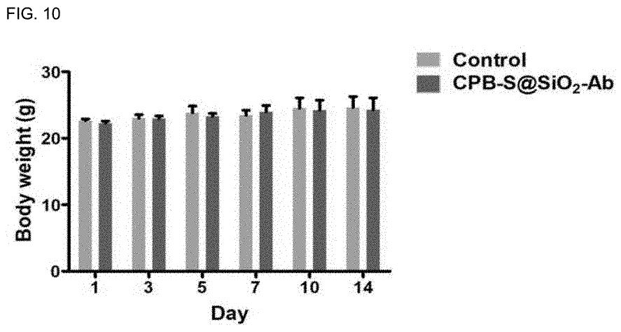

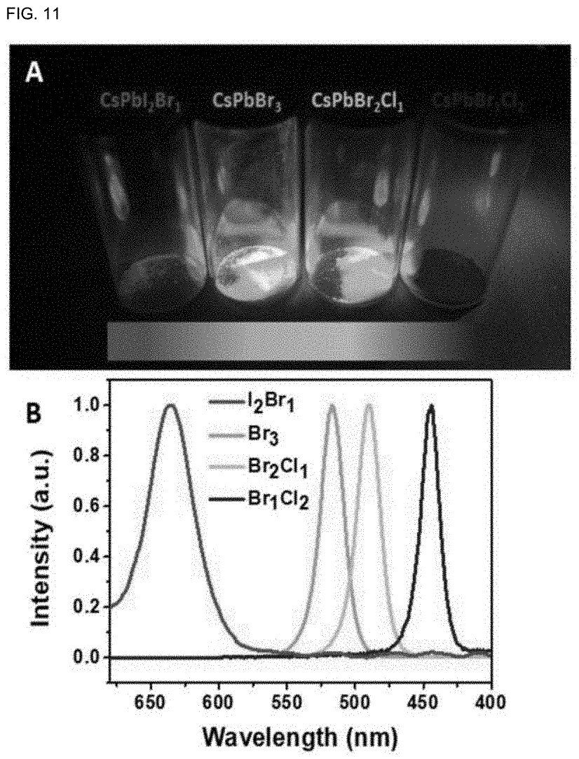

A new and effective synthetic method was developed to embed CsPbX₃ QDs inside SiO₂ NPs, namely the rapid co-synthesis of QDs and NPs. After rapidly injecting a QD precursor solution containing the base catalyst for SiO₂ synthesis into a Si precursor solution, base-catalyzed synthesis of SiO₂ NPs and ligand-assisted re-precipitation (LARP) of CsPbX₃ QDs occurred simultaneously in a very short time. This rapid co-synthesis resulted in the CsPbX₃ QDs being trapped inside the SiO₂ NPs before their decomposition occurred. FIG. 11 shows the synthesis results of various types of CsPbX₃-SiO₂@SiO₂ NP powders according to the halogen composition, (A) a photograph of each powder under UV irradiation and (B) the photoluminescence measurement results of each powder. The molecular formula according to the halogen (X) composition of each CsPbX₃ quantum dot is CsPbl₂Br₁ (RED), CsPbBr₃ (GREEN), CsPbBr₂Cl₁ (CYAN), CsPbBr₁Cl₂ (BLUE), and exhibited unique photoluminescence according to the change of X. In addition, post-annealing at 150° C. significantly reduced the surface hydroxyl groups, as indicated in FT-IR spectra by the reduction of the Si-OH stretching peak at 950 cm⁻¹ (FIG. 2A). In the binding energy region of O 1 s electrons, XPS measurements also showed a reduction of the surface hydroxyl groups (FIGS. 2B and 2C). The area of the Si-OH-related O 1 s peak at 533.2 eV was reduced to 76.4% after annealing, consistent with the FT-IR results (FIG. 2D). The annealing and consequently reduced number of hydroxyl groups contributed to the stability of the CPB QDs inside, as shown by the change in photoluminescence (PL) intensity over time (FIG. 3). The non-annealed CPB-SiO₂ NPs rapidly lost their PL properties, reaching only one-fifth of initial PL intensity after 350 h. The annealed CPB-SiO₂ NPs were then encapsulated once again by growth of additional SiO₂ layers on the NPs to more securely block the release of CPB QDs or any other decomposition products. The TEM images clearly show the resulting CPB-SiO₂@SiO₂ core-shell structure (FIGS. 1C and 1D). The average diameter of the CPB-SiO₂ core was 100 nm and the thickness of the SiO₂ shell layer was approximately 26 nm. CPB QDs of 8-11 nm size were clearly observed in the magnified TEM image, as marked with arrows in FIG. 10. The 512 nm-wavelength green emission of the NPs synthesized under UV irradiation, characteristic of 4-15 nm size CPB QDs, indicated that the QDs exhibit their PL characteristics well even within SiO₂ NPs (FIG. 1E and FIG. 4).

### <Experimental Example 2> Stability of CPB-SiO₂@SiO₂ NPs

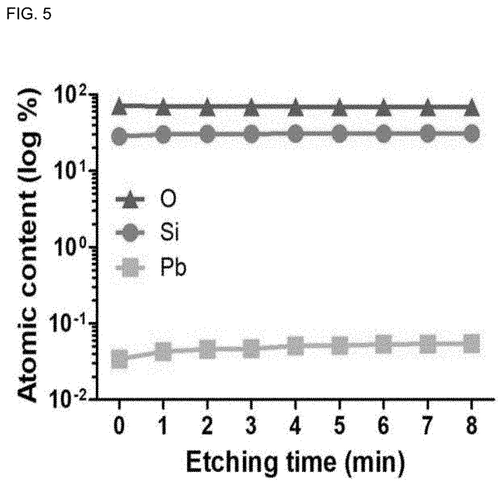

The embedded amounts of CPB QDs in the CPB-SiO₂@SiO₂ NPs was estimated by depth-profile XPS measurements of the spin-coated NP thin films (FIG. 5). Shortly after etching, the atomic contents of Si, O and Pb atoms became almost constant at 31.1%, 68.8% and 0.046%, respectively, indicating that the total quantity of Pb atoms was approximately 0.5 mg per 1 g of NPs. The amount of NPs IV-injected in the present invention (200 μg) corresponds to a total mass of only 1 μg of Pb. The blood lead content caused by the CPB QDs in the NPs will be even less than this because the QDs are very stable inside the NPs. FIG. 2E shows the variation of Pb concentration over time, estimated from inductively coupled plasma (ICP) measurements for aqueous CPB-SiO₂@SiO₂ NP solution (1 mg/ml). Even after 14 days, detected Pb concentration in the solution was only 0.2 ppb (0.02 μg/dI), indicating that Pb or Pb-related compounds are released from the NPs in only tiny quantities. The observed Pb contents are much lower than the maximum blood lead levels recommended by the World Health Organization (WHO) and the Centers for Disease Control and Prevention (CDC) of 10 μg/dl for adults and 5 μg/dl for children. The stability of the CPB QDs in the NPs was also confirmed by PL measurements (FIG. 2F). The PL intensity of the 1.0 mg/mI CPB-SiO₂@SiO₂ NP solution remained nearly unchanged for five days and decreased only slightly to 92% of the initial value after fourteen days.

### <Experimental Example 3> X-ray Attenuation Characteristic of CPB-SiO₂@SiO₂ NPs

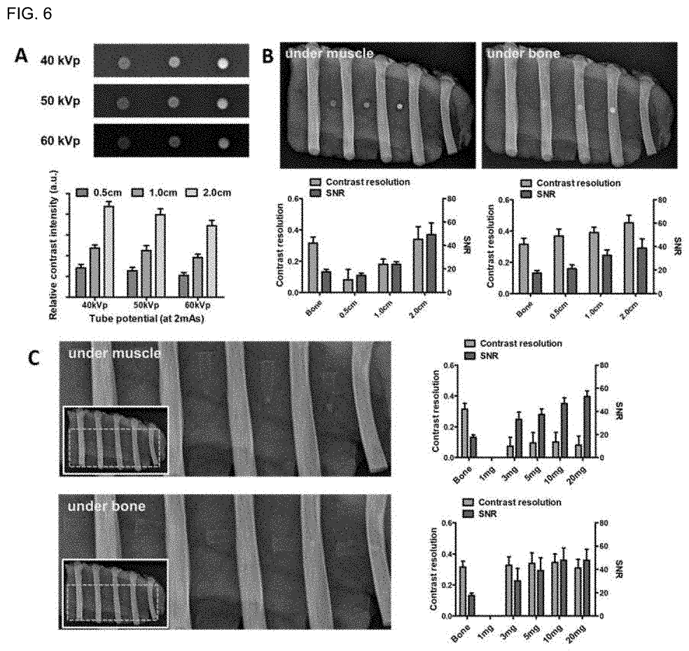

The X-ray attenuation by the CPB-SiO₂@SiO₂ NPs was evaluated using clinical X-ray equipment (EVA-HF520, COMED). Radiographic images of the NPs-containing plastic cylinder with a thickness of 0.5, 1.0 and 2.0 cm were acquired at various X-ray tube potentials from 40, 50, 60 kVp (peak kilovoltage) (FIG. 6A). The X-ray beam intensity and source-image distance (SID) were fixed at 2 mAs (milliampere-second) and 100 cm, respectively. The brighter image, indicative of higher X-ray attenuation, as the sample thickness increased was observed for each tube potential. To ensure that this observation of X-ray attenuation is also valid deep inside the human body, an alternative experiment using pork ribs with 300 g weight and 1.5 cm thickness was conducted. As shown in FIG. 6B, the CPB-SiO₂@SiO₂ NPs placed under muscle or bone were apparently identified by X-ray examination of the tube voltage fixed to 50 kVp. Under muscle, the contrast resolution of the 2.0 cm-thick NPs was 0.34, which was slightly larger than that of bone, 0.32. The contrast resolution is defined as (S_{ROI}-S_{muscle})/(S_{ROI}S_{muscle}) where S_{ROI} and S_{muscle} are signal intensities of the region of interest (ROI) and muscle, respectively. Although contrast resolutions of the 0.5 and 1.0 cm-thick NPs, 0.08 and 0.18, respectively, were smaller than that of the 2.0 cm-thick NPs, they were also easily distinguishable since the human eye can detect a minimum contrast of about 0.005 to 0.05. The contrast resolutions of the NPs under bone were all larger (0.37, 0.39 and 0.45) than that of the bone (0.32), indicating that the X-ray attenuation by the NPs and bone was combined. The signal to noise ratio (SNR), defined as the ratio of the average intensity to standard deviation of the signal, was also evaluated. The SNR of the NPs except 0.5 cm-thick NPs under muscle, were larger than that of the bone, indicating that the NPs glow more uniformly, compared to the bone. All these results indicate that the synthesized CPB-SiO₂@SiO₂ NPs can be distinctly identified by plain X-ray imaging, even they are hidden in the tissues such as muscles and bones. A significantly small amount of the NPs were also tested to determine the minimum dose to be recognized in the X-ray radiography (FIG. 6C). While it was difficult to distinguish the contrast between the 1 mg NPs and muscle, the NPs over 3 mg (equivalent to 10 mg/kg tissue weight) were clearly distinguishable. The SNR of the NPs were all larger than that of the bone.

### <Experimental Example 4> CPB-SiO₂@SiO₂ NPs Uptake in Vitro and Cell Viability Assay

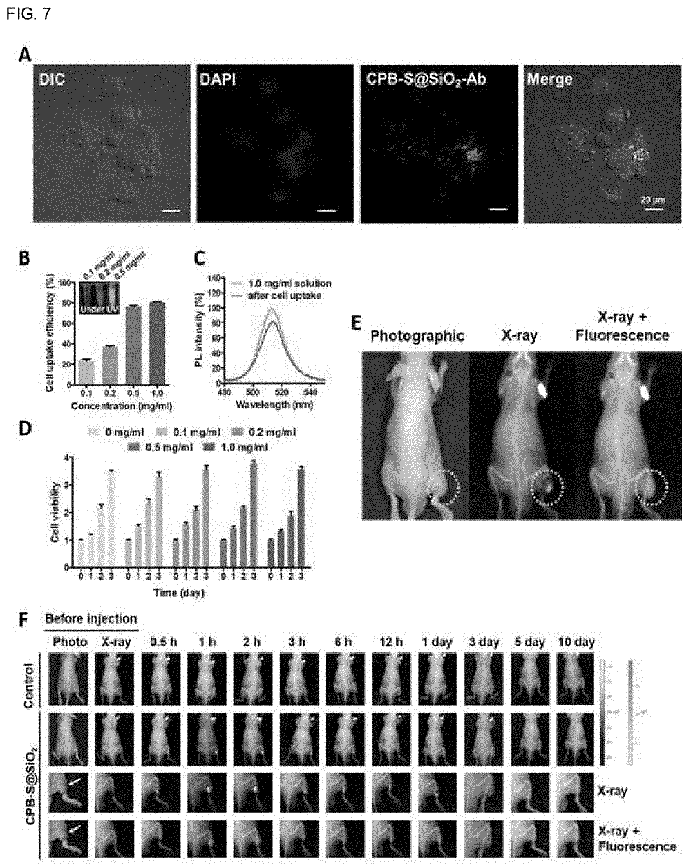

To target the CD44 surface adhesion receptor of the pancreatic cells, the surface of the synthesized CPB-SiO₂@SiO₂ NPs was modified with anti-CD44 antibodies. Because its expression is generally associated with a poor prognosis, CD44 is an important prognostic marker and therapeutic target of pancreatic cancer. The anti-CD44 antibodies were conjugated on the surface of the CPB-SiO₂@SiO₂ NPs by reacting the maleimide-activated NP surface with sulfhydryl groups on the antibodies. The uptake of CPB-SiO₂@SiO₂-Ab NPs in Panc-1 cells was evaluated by confocal laser-scanning microscopy (CLSM) (FIG. 7A). The nuclei of the cells were stained with 4, 6-diamidino-2-phenylindole (DAPI), emitting in the blue range, maximally at 461 nm. Bright green fluorescence from the CPB QDs was observed, mostly in non-blue areas, indicating that the QDs are stable in the cells and mainly localized in the cytosol.

To verify the uptake efficiency of the CPB-SiO₂@SiO₂-Ab NPs, Panc-1 cells were seeded at a density of 1×10⁶ on a 60-mm cell culture plate and held for 24 h; this was followed by treatment with various concentrations of NPs (0, 0.1, 0.2, 0.5 and 1 mg/ml) for another 24 h, after which cells were fixed with formaldehyde. The PL intensity of the Panc-1 cells, indicative of CPB QD uptake, increased steadily as the concentration of NPs increased to 0.5 mg/ml, but showed no apparent increase thereafter (FIG. 7B). For the 1 mg/ml solution, a maximum uptake of 76.8% was obtained (FIG. 7C). Study on the cytotoxicity of CPB-SiO₂@SiO₂-Ab NPs is very important for their application to cancer imaging. FIG. 7D shows the effect of CPB-SiO₂@SiO₂-Ab NPs on the viability of Panc-1 cells, as observed through WST-1 assay. For all concentrations of NPs, no cytotoxicity was observed under cell culture condition, and no other effect on cell proliferation or differentiation was observed. This nontoxicity of the CPB-SiO₂@SiO₂-Ab NPs clearly indicated that very little Pb was released from the NPs because Pb-induced cell death has been reported when Pb concentration is larger than 3 μM.

### <Experimental Example 5> In Vivo X-Ray Cancer Imaging

When the transplanted Panc-1 cells had grown to a sufficient volume in the xenograft mouse, 200 μl of 1 mg/ml CPB-SiO₂@SiO₂-Ab NP solution, equaling an NP dose of 10 mg/kg body weight, was injected intravenously. FIG. 7E clearly shows the white and green signals at the tumor position of the xenograft mouse under X-ray irradiation. This indicates that the antibody-conjugated NPs successfully recognized the CD44 prognostic markers and, more importantly, that the cancer can be easily detected in vivo by simple plain X-ray imaging. Because the tumor was grown immediately below the skin, the green X-ray-induced fluorescence was also detected due to the well-known capability of CPB QDs to emit radiation at a wavelength of 512 nm.

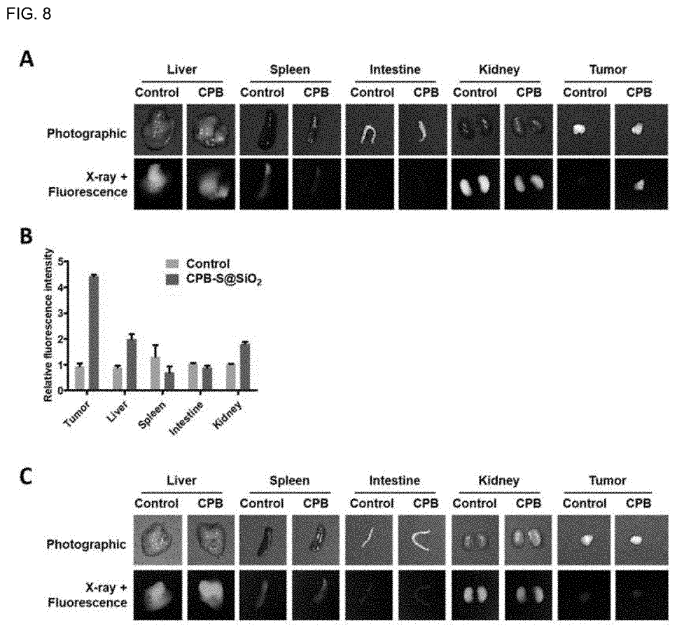

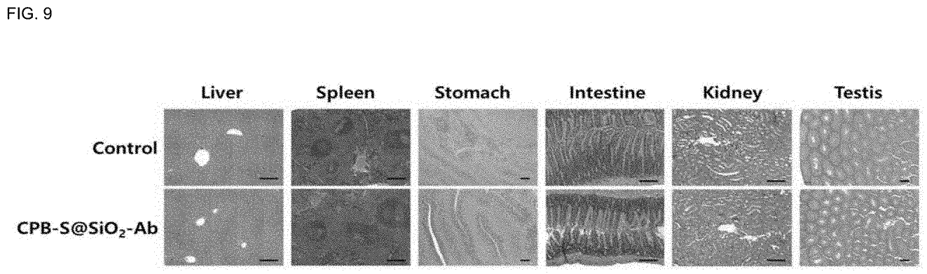

Real-time biodistribution of injected CPB-SiO₂@SiO₂-Ab NPs was monitored through X-ray and fluorescence imaging, with results shown in FIG. 7F. The intensity of the signals gradually increased over time, reaching a maximum 2 h after the injection; after 5 days, no detectable signals were observed. To investigate the biodistribution in detail, fluorescent images were taken of various organs dissected 2 h and 10 days after NP injection (FIG. 8). As expected, 2 h after injection (FIFs. 8A and 8B), an intense green emission was detected in the approximately 5 mm diameter tumor. The fluorescence intensity was 4.4 times higher than that of the untreated control cells. Although the fluorescence intensities in the liver and kidney were also slightly higher than they were in the case without NPs, no apparent intensity change was observed in the spleen and intestine. The absence of fluorescence 10 days after NP injection indicates that no CPB QDs were left in any organ (FIG. 8C). To evaluate acute toxicity of CPB-SiO₂@SiO₂-Ab NPs, hematoxylin and eosin (H&E) staining of various organs from the NP-treated mice was performed (FIG. 9). No discernible differences were observed in the morphology of tissues, compared to the organs from untreated control mice, indicating the biocompatibility of the NPs. Changes of body weight was monitored as a basic measure of toxicity evaluation, but no significant weight difference was observed among the treatment groups (FIG. 10).

Therefore, it was confirmed that cancer can be efficiently and in real time detected by a non-invasive plain X-ray examination without cytotoxicity using CPB QD stably confined in SiO₂ NP.

The nanoparticles for real-time biological imaging based on X-ray attenuation according to the present invention can be rapidly prepared by co-synthesis, and have excellent safety and stability in vivo.

In addition, it has an effect of enabling real-time biometric imaging such as simple and rapid early diagnosis of cancer by plain X-ray imaging, such as those used for non-invasive chest X-ray.

What is claimed is:

1. Nanoparticles for biological imaging based on X-ray attenuation comprising:
a core structure comprising an X-ray attenuating material; and
a shell layer formed on the core structure and made of a material having biocompatibility and non-reactivity in vivo.

2. The nanoparticles for biological imaging based on X-ray attenuation of claim 1, the X-ray attenuating material is quantum dot having an average diameter of 5 to 15 nm.

3. The nanoparticles for biological imaging based on X-ray attenuation of claim 2, the quantum dot is ABX₃ perovskite structure material,
wherein the A is selected from the group consisting of Ti, Sr, Ca, Cs, Ba, Y, Gd, La, Fe and Mn, the B is selected from the group consisting of Pb, Sn, Cu, Ni, Bi, Co, Fe, Mn, Cr, Cd, Ge and Yb, and the X is selected from the group consisting of l_{y}Br_{(1-y)}, l_{y}Cl_{(1-y)} and Br_{y}Cl_{(1-y)} (0≤y≤1)).

4. The nanoparticles for biological imaging based on X-ray attenuation of claim 1, the shell layer is at least one selected from the group consisting of SiO₂, TiO₂, ZnO, ZrO₂ and Al₂O₃.

5. The nanoparticles for biological imaging based on X-ray attenuation of claim 1, the shell layer further comprises at least one targeting agent selected from the group consisting of enzyme substrate, ligand, amino acid, peptide, protein, nucleic acid, lipid, cofactor, carbohydrate and antibody on the surface thereof.

6. A composition for biological imaging based on X-ray attenuation comprising the nanoparticles for biological imaging based on X-ray attenuation of claim 1 as an active ingredient.

7. A method of biological imaging based on X-ray attenuation comprising:
reacting the nanoparticles for biological imaging based on X-ray attenuation of claim 1 with a biological sample; and
observing an image by X-ray imaging.

8. A method of preparing nanoparticles for biological imaging based on X-ray attenuation comprising:
preparing an X-ray attenuating material precursor solution (Step 1);
preparing a core structure by adding the solution of the step 1 to a core precursor solution (Step 2);
annealing the core structure of the step 2 (Step 3); and
preparing nanoparticles having a core precursor-shell layer by adding annealed core structure of the step 3 to a shell precursor solution (Step 4).

9. The method of preparing nanoparticles for biological imaging based on X-ray attenuation of claim 8, wherein the X-ray attenuating material precursor is selected from the group consisting of lead bromide (PbBr₂), cesium bromide (CsBr), cesium iodide (Csl), cesium chloride (CsCl) and lead chloride (PbCl₂).

10. The method of preparing nanoparticles for biological imaging based on X-ray attenuation of claim 8, wherein the X-ray attenuation material precursor solution further comprises a basic catalyst.

11. The method of preparing nanoparticles for biological imaging based on X-ray attenuation of claim 8, wherein the core precursor or the shell precursor is selected from the group consisting of tetramethyl orthosilicate and tetraethyl orthosilicate.

12. The method of preparing nanoparticles for biological imaging based on X-ray attenuation of claim 8, wherein the annealing is performed at 100 to 200° C. for 1 to 3 hours.

13. The method of preparing nanoparticles for biological imaging based on X-ray attenuation of claim 8, further comprising binding a targeting agent to the shell layer of the nanoparticles having the core precursor-shell layer.

14. The method of preparing nanoparticles for biological imaging based on X-ray attenuation of claim 13, wherein the binding the targeting agent to the shell layer of the nanoparticles having the core precursor-shell layer comprises:
modifying the shell layer by reacting the nanoparticles having the core precursor-shell layer with 3-aminopropyl triethoxysilane and 4-(N-maleimidomethyl)cyclohexane-1-carboxylic acid 3-sulfo-N-hydroxysuccinimide ester sodium salt; and
conjugating modified nanoparticles with a targeting agent.
